(19) United States
(12) Patent Application Publication
(10) Pub. No.: US 2025/0268753 A1
(43) Pub. Date: Aug. 28, 2025

(54) INTELLIGENT DETECTION AND TREATMENT OF ISCHEMIC TISSUE

(71) Applicant: Alcon Inc., Fribourg (CH)

(72) Inventor: Paul R. Hallen, Colleyville, TX (US)

(21) Appl. No.: 19/015,938

(22) Filed: Jan. 10, 2025

Related U.S. Application Data
(60) Provisional application No. 63/556,490, filed on Feb. 22, 2024.

(51) Int. Cl. A61F 9/008

(52) U.S. Cl. CPC A61F 9/008; A61F 2009/00844; A61F 2009/00863; A61F 2009/00885

(57) ABSTRACT

In an embodiment, a surgical system for treating ischemic tissue includes an oxygenation sensor, a laser source operable to emit treatment laser light, and a processor in communication with the oxygenation sensor and the laser source. The processor is operable to cause the oxygenation sensor to detect an oxygenation status of a tissue based on illumination light reflected from the tissue. The processor is further operable to generate a feedback signal related to utilization of the laser source in relation to the tissue based on the oxygenation status. Other embodiments of this aspect include corresponding computer systems, apparatus, and computer programs recorded on one or more computer storage devices, each configured to perform the actions of the methods.

### INTRODUCTION

Ischemic tissue in the retina can develop when insufficient oxygen reaches the tissue. This lack of oxygen causes the death of cells as the tissue becomes denatured in its deoxygenated state. A buildup of ischemic tissue can result in vison loss, among other complications.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by a data processing apparatus, cause the apparatus to perform the actions.

In an embodiment, one general aspect involves a surgical system for treating ischemic tissue. The surgical system includes an oxygenation sensor, a laser source operable to emit treatment laser light, and a processor in communication with the oxygenation sensor and the laser source. The oxygenation sensor is operable to detect an oxygenation status of a tissue based on illumination light reflected from the tissue. The processor is operable to generate a feedback signal related to utilization of the laser source in relation to the tissue based on the oxygenation status.

In an embodiment, another general aspect involves a method for treating ischemic tissue. The method includes detecting, by an oxygenation sensor, an oxygenation status of a tissue based on illumination light reflected from the tissue. The method further includes generating, at a processor, a feedback signal related to utilization of a laser source in relation to the tissue based on oxygenation status.

### BRIEF DESCRIPTION OF THE DRAWINGS

For a more complete understanding of the present technology, its features, and its advantages, reference is made to the following description, taken in conjunction with the accompanying drawings, in which:

FIG. 1 illustrates a surgical console that may be used with systems and methods in accordance with certain embodiments of the present disclosure.

FIG. 2A illustrates a system for providing illumination light and/or a laser light to a surgical target, in accordance with certain embodiments of the present disclosure.

FIG. 2B illustrates a detection and treatment system, in accordance with certain embodiments of the present disclosure.

FIG. 3 illustrates an example of a process for treating ischemic tissue, in accordance with certain embodiments of the present disclosure.

FIG. 4 illustrates an example of a process for generating a feedback signal in an automatic mode, in accordance with certain embodiments of the present disclosure.

FIG. 5 illustrates an example of a process for generating a feedback signal in a guided mode, in accordance with certain embodiments of the present disclosure.

FIG. 6 illustrates an example of an operating environment for performance of a surgical procedure, such as treatment of ischemic tissue, in accordance with certain embodiments of the present disclosure.

### DETAILED DESCRIPTION

In the following description, details are set forth by way of example to facilitate an understanding of the disclosed subject matter. It should be apparent to a person of ordinary skill in the field, however, that the disclosed implementations are exemplary and not exhaustive of all possible implementations. Thus, it should be understood that reference to the described example is not intended to limit the scope of the disclosure. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure.

Ischemic tissue, sometimes referred to herein as deoxygenated tissue, can develop in the retina when insufficient oxygen reaches the tissue. This lack of oxygen causes the death of cells as the tissue becomes denatured in its deoxygenated state. A buildup of ischemic tissue can result in vison loss, among other complications. One possible treatment method involves a patient ingesting Fluorescein dye, followed by a surgeon locating ischemic tissue indicated by the dye and irradiating it. This method involves a two-step process of first detection, followed by a separate second step of treatment (e.g., irradiation), as well as an element of invasiveness, as the patient must actively partake in ingesting a substance that momentarily alters the state of his or her body.

The present disclosure describes examples of surgical systems and methods for intelligently detecting and treating ischemic tissue using, for example, laser technology. In various embodiments, laser application can be limited to the ischemic tissue. For example, in some embodiments, treatment laser light can be transmitted automatically in response to detection of ischemic tissue. In addition, or alternatively, real-time feedback can be provided to operators to facilitate manual transmission of treatment laser light. Examples will be described relative to the Drawings.

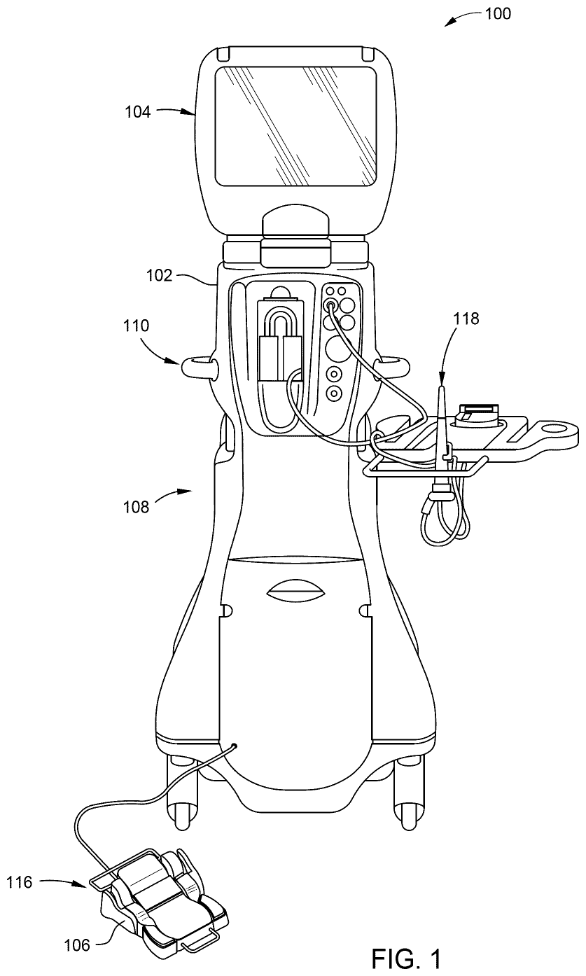

FIG. 1 depicts a surgical console 100 that may be used with systems and methods in accordance with certain embodiments of the present disclosure. Generally, the example surgical console 100 may be similar to ophthalmic surgical consoles that have been known and used, such as the CONSTELLATION® Vision System available from Alcon Laboratories, Inc. (Fort Worth, Texas) or the CENTURION® Vision System available from Alcon Laboratories, Inc. (Fort Worth, Texas), or any other ophthalmic surgical console suitable for use with the principles described herein.

As shown in FIG. 1, the surgical console 100 includes a housing 102 with a computer system 108 integrated therewith and an associated external display 104. In some embodiments, data relating to system operation and performance is displayed on the external display 104 during an ophthalmic surgical procedure. The surgical console 100 is further in wired or wireless communication with a foot pedal 106 that an operator may use in controlling one or more functions and/or systems of the surgical console 100, and a surgical probe 118 for performing various operations of the procedure. The surgical console 100 may also include one or more systems and/or subsystems that may be used while performing an ophthalmic surgical procedure, such as an ischemic tissue treatment procedure. For example, the surgical console 100 may include a foot pedal system 116 in wired or wireless communication with the foot pedal 106.

The external display 104 of the present disclosure, in some embodiments, is configured to connect to probe 118 via an electrical cable, enabling signals to be displayed to the surgeon communicated to the external display 104 by the feedback signal generated by the processor.

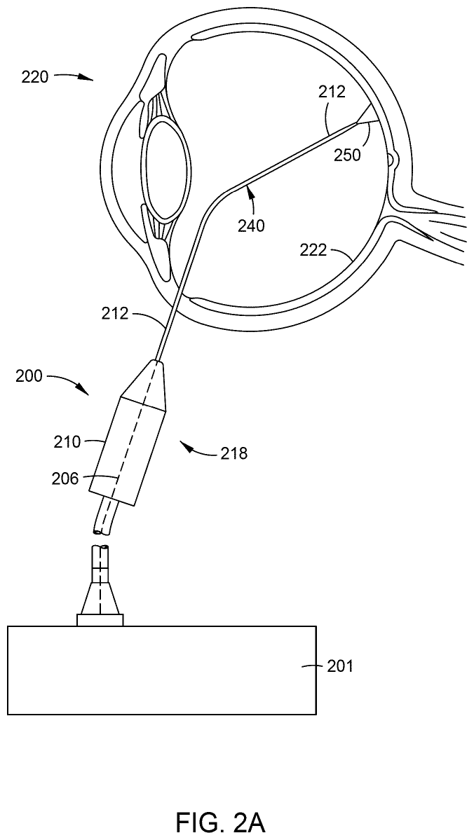

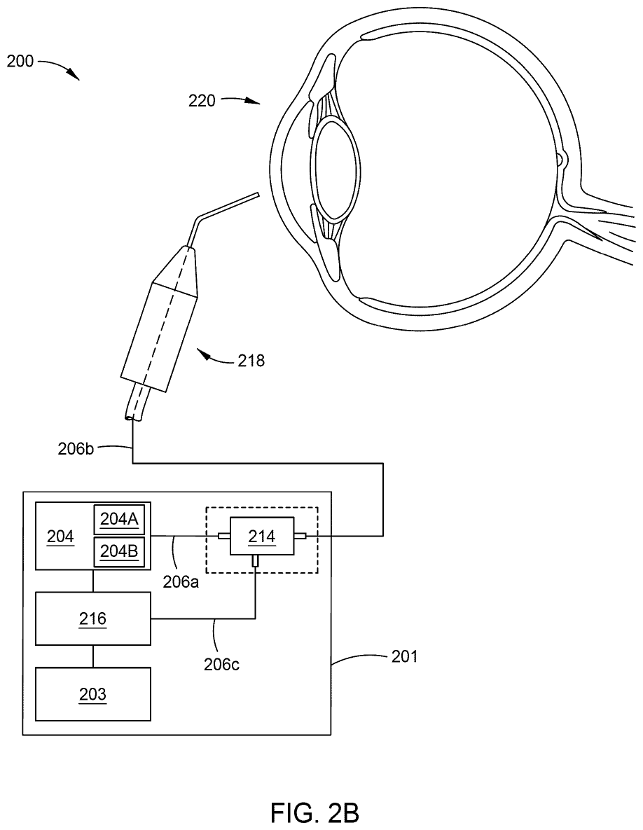

FIG. 2A illustrates a system 200 for providing an illumination light and/or a laser light to a surgical target, in accordance with certain embodiments of the present disclosure. As shown, system 200 includes a detection and treatment system 201 and a probe 218. Probe 218 may be the same as or similar to probe 118 shown in FIG. 1. Detection and treatment system 201 may include, or be part of, surgical console 100 shown in FIG. 1. Detection and treatment system 201 may include one or more light sources (e.g., laser and/or illumination light sources) for generating laser light beams and/or illumination light beams that may be used during an ophthalmic procedure. For example, the light sources may alternatively, sequentially, or simultaneously generate a laser light beam and an illumination light beam. A user, such as a surgeon or surgical staff member, may control detection and treatment system 201 (e.g., via foot pedal such as the foot pedal 106 of FIG. 1, voice commands, etc.) to emit the laser light beam and/or the illumination light beam during an ophthalmic procedure, such as vitreoretinal surgery.

System 200 delivers the laser and/or illumination light beams to probe 218 via optical fiber 206. As shown, probe 218 includes a hand-piece, or probe body, 210. Probe 218 also includes a probe tip 240 coupled to a distal end of hand-piece 210. Note that, herein, a distal end of a component refers to the end that is closer to a patient's body, or where the laser and/or illumination light is emitted out of the probe. On the other hand, the proximal end of the component refers to the end that is facing away from the patient's body or in proximity to, for example, the light source. Probe tip 240 includes a tube 212 extending an entire length of probe tip 240. In certain embodiments, tube 212 is a cylindrical hollow tube. A distal end and a proximal end of probe tip 240 and thus, of tube 212, are depicted in FIG. 1. Although not shown herein, optical fiber 206 extends an entire length of tube 212 to transmit laser and/or illumination light to the distal end of tube 212.

In operation, a surgeon uses hand-piece 210 to guide tube 212 into a patient's eye 220. Tube 212 is only partly inserted into eye 220 such that the proximal end of tube 212 is disposed outside eye 220. A laser and/or illumination light source of detection and treatment system 201 generates a light beam 250, which is directed by tube 212 to a desired location/surface of eye 220, such as retinal surface 222. In certain embodiments, probe 218 is a multi-spot laser probe and concurrently provides multiple laser light beams 250 resulting in multiple laser spots. In some embodiments, each laser spot's power may be within a range of about 150 milliwatts (mW) to about 500 mW such that by providing multiple laser spots, the minimum power passing through tube 212 is about 1 Watt (W). In certain embodiments, a lens is positioned in front of the one or more optical fibers in tube 212 for projecting the laser and/or illumination light beams onto the desired location of eye 220. Thus, as described above, system 200 is capable of projecting a laser and/or illumination light beam onto a desired location of the eye during a surgical procedure, e.g., retinal laser treatment.

FIG. 2B illustrates the system 200 of FIG. 2A in greater detail, according to certain embodiments of the present disclosure. Detection and treatment system 201 generally includes light sources 204, an oxygenation sensor 216, an optical circulator 214, and a processor 203. As shown in FIG. 2A, optical circulator 214 is optically coupled to each of light source 204, oxygenation sensor 216, and probe 218 through multiple optical fibers 206 (206 a-c). In the illustrated embodiments, optical fiber 206a is coupled between light source 204 and optical circulator 214, optical fiber 206b is coupled between optical circulator 214 and probe 218, and optical fiber 206c is coupled between oxygenation sensor 216 and optical circulator 214. Together, optical fibers 206 a-c enable transmission of laser and/or illumination light from light source 204 to probe 218 and from probe 218 to oxygenation sensor 216. In certain embodiments, optical circulator 214 is directly coupled to one or both of light source 204 or oxygenation sensor 216 such that optical system 200 may operate without one or both of optical fibers 206a or 206b. Directly coupling oxygenation sensor 216 to optical circulator 214 may improve photodetection by reducing overall loss of the return light.

In the illustrated embodiments, optical fiber 206b is disposed inside probe 218. In certain embodiments, optical fiber 206b may extend an entire length of probe 218 to transmit laser and/or illumination light therethrough. In some embodiments, the system 200 is able to function as described herein when optical fiber 206b includes only one optical fiber. For example, a single optical fiber may transmit both laser light and illumination light. However, in some other embodiments, probe 218 includes two or more optical fibers to provide added functionality. In certain embodiments, a first optical fiber housed in probe 218 transmits an illumination light from a corresponding illumination source while a second optical fiber housed in probe 218 transmits a laser light from a corresponding laser source.

Light sources 204 may include, for example, a treatment laser source 204A (coherent light source) and/or an illumination source 204B (incoherent light source). The treatment laser source 204A can transmit, for example, treatment laser light to irradiate ischemic tissue without damaging surrounding tissue. The treatment laser source 204A can transmit a laser which can be of any desired wavelength, such as from about 532 nm (nanometers) to about 635 nm.

In certain embodiments, illumination source 204B is a xenon-based or LED-based (light emitting diode based) illuminator. In certain embodiments, illumination source 204B is a broadband light source or hyperspectral light source. Hyperspectral light may include light beyond the visible spectrum including, e.g., infrared and ultraviolet light. Other light sources are also contemplated. For example, instead of a broadband light source, narrowband/discrete light source(s) (such as blue, green, red, etc. light) may be used for multispectral imaging. In certain embodiments, illumination source 204B is integrated with a console (e.g., surgical console 100 of FIG. 1). In some other embodiments, illumination source 204B is a stand-alone light source.

In certain embodiments, oxygenation sensor 216 is operable to detect an oxygenation status of tissues or structures based on illumination light emitted from the illumination source 204B to the tissues or structures. In various embodiments, the oxygenation sensor 216 can generate oxygenation data that includes, for example, an identification of the detected oxygenation status. For example, oxygenation sensor 216 can cause the illumination source 204B to emit the illumination light to the tissues or structures and thereafter collect return light reflected from the targeted tissues/structures for subsequent analysis. In some embodiments, such as embodiments in which the illumination source 204B utilizes hyperspectral light, the oxygenation sensor 216 can generate hyperspectral image data on the tissues or structures and, based thereon, detect the oxygen based on changes in total hemoglobin levels in the tissues or structures.

In certain embodiments, the processor 203 can receive the oxygenation data from the oxygenation sensor 216 and generate a feedback signal related to utilization of the treatment laser source 204A in relation to the tissue (e.g., on the tissue) based thereon (e.g., based on the oxygenation status). The feedback signal can include, for example, an instruction to the treatment laser source 204A to transmit treatment laser light to the tissue, a signal to an operator (e.g., a surgeon or surgical staff member) to manually cause transmission of the treatment laser light (e.g., via a foot pedal such as the foot pedal 106 of FIG. 1), and/or the like. In some embodiments, the processor 203 utilizes machine learning to decipher the oxygenation data and produce the feedback signal. Different methods can be used to assess the oxygenation status of the tissue. The data produced by the oxygenation sensor 216 is fed to the processor 203 wherein the types of analysis it performs on the hyperspectral images includes but is not limited to principal component analysis, K-means clustering, Gaussian superposition, Principal Component Analysis, among other assessment techniques.

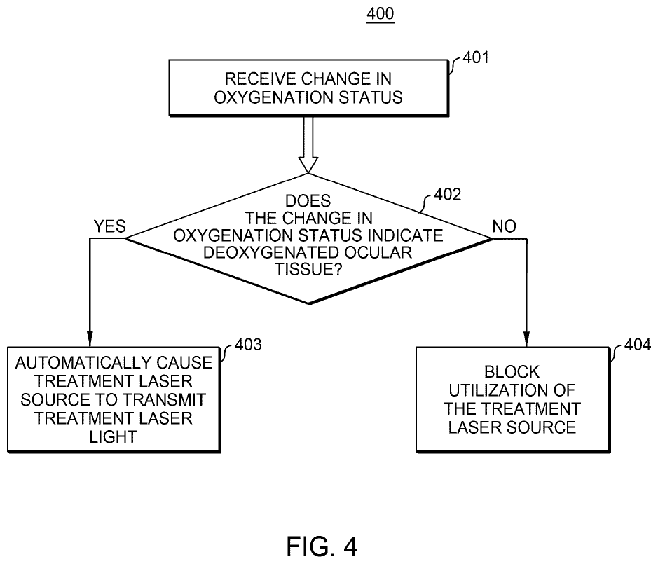

In various embodiments, the processor 203 can generate the feedback signal in accordance with its operational mode. In some embodiments, the detection and treatment system 201 can operate in an automatic mode in which deoxygenated (i.e., ischemic) tissue is automatically irradiated. For example, in the automatic mode, the processor 203 can automatically, without operator intervention, cause (e.g., instruct) the treatment laser source 204A to transmit treatment laser light to the deoxygenated tissue. In these embodiments, the automatic causing (e.g., an instruction to the treatment laser source 204A) can constitute, at least in part, the feedback signal generated by the detection and treatment system 201. An example of the automatic mode will be described in greater detail relative to FIG. 4.

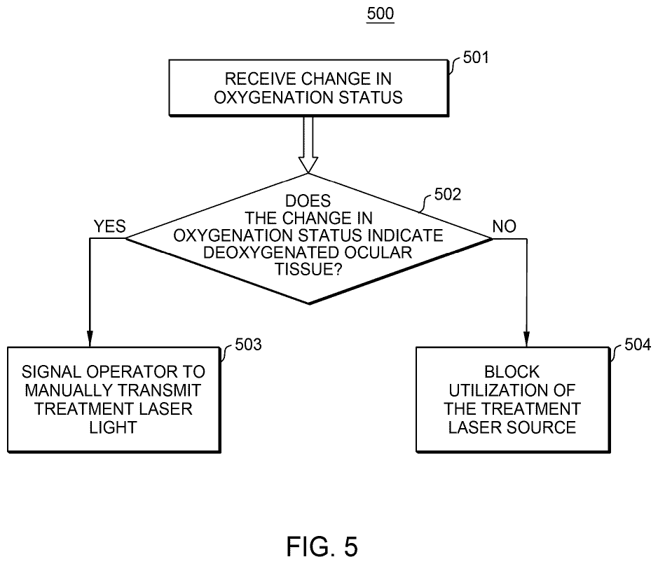

In addition, or alternatively, in some embodiments, the detection and treatment system 201 can operate in a guided mode in which the operator is prompted to manually cause irradiation of the deoxygenated tissue. For example, in the guided mode, the processor 203 can signal the operator (e.g., via visual, auditory, and/or haptic feedback) to manually transmit treatment laser light to the deoxygenated tissue. In these embodiments, the signal to the operator can constitute, at least in part, the feedback signal generated by the detection and treatment system 201. In some embodiments, the signal to the operator can be auditory via the probe 218 and/or a surgical console such as the surgical console 100 of FIG. 1, visual on the probe 218 and/or on a display such as the display 104 of FIG. 1, and/or the like. In addition, or alternatively, the signal to the operator can be haptic feedback that is felt, for example, on the probe 218, on a foot pedal such as the foot pedal 106 of FIG. 1, and/or the like. An example of the guided mode will be described in greater detail relative to FIG. 5.

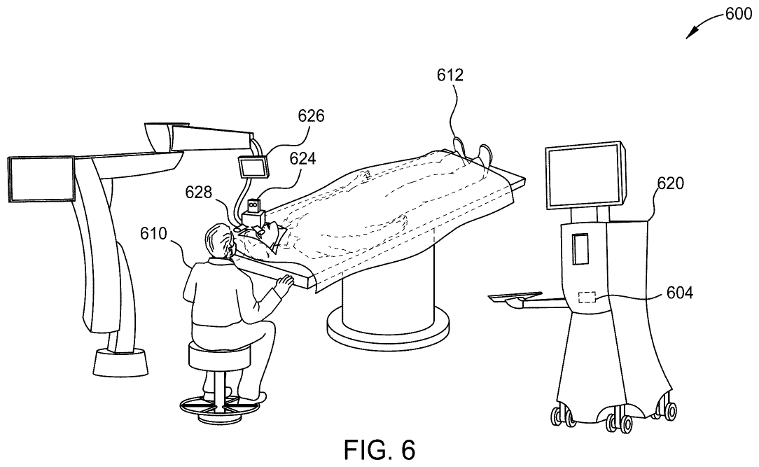

In some embodiments, in the automatic mode and/or in the guided mode, the operator can be presented real-time information on a display such as the display 104 of FIG. 1 or in a microscope system, as more specifically shown and described to FIG. 6. For example, the processor 203 can generate and cause display of a real-time retinal map that indicates, for example, an area of the retinal surface 222 corresponding to a current location of the distal end of probe tip 240. The retinal map can be generated by combining, for example, 580 nm and 590 nm wavelength images. In some cases, the retinal map can be pre-processed by the processor 203, prior to presentation to the operator. In these cases, the dual wavelength technique can provide data to the processor 203 for evaluating arteries and veins to further determine information related whether the tissue is deoxygenated. In some embodiments, the processed and/or displayed information can further include any collected hyperspectral and/or multispectral image data referenced above, such as multispectral images where deeper oxygen saturation of the ocular tissue is represented by generated mean scale values.

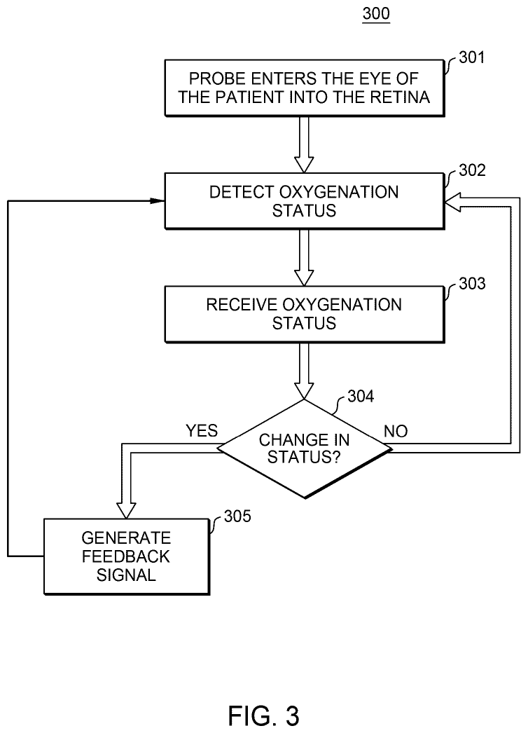

FIG. 3 illustrates an example of a process 300 for treating ischemic tissue in accordance with certain embodiments of the present disclosure. For illustrative purposes, the process 300 will be described relative to the system 200 described relative to FIGS. 2A-B.

At block 301, the probe 218 is caused, for example, by an operator, to enter the eye 220 as described relative to FIGS. 2A-B. At block 302, the oxygenation sensor 216 detects an oxygenation status of ocular tissue, for example, on the retinal surface 222, in correspondence to the operator's control of the probe 218. In some embodiments, the block 302 can include the processor 203 causing the oxygenation sensor 216 to perform the detection. At block 303, the processor 203 receives the oxygenation status detected by the oxygenation sensor 216.

At decision block 304, the processor 203 determines whether there has been a change in oxygenation status indicated, for example, by the oxygenation sensor 216. If not, the process 300 returns to the block 302, where the oxygenation sensor 216 continues detecting an oxygenation status of ocular tissue as described previously. Otherwise, if it is determined, at the decision block 304, that there has been a change in oxygenation status indicated, for example, by the oxygenation sensor 216, the process 300 proceeds to block 305.

At block 305, the processor 203 generates a feedback signal related to utilization of the treatment laser source 204A in relation to the tissue. In general, the feedback signal can be generated in any of the ways described relative to FIGS. 2A-B. For example, depending on the oxygenation status of the ocular tissue, the feedback signal can include, for example, an automatic instruction to the treatment laser source 204A, a signal to the operator regarding the oxygenation status, and/or the like. As described relative to FIGS. 2A-B, in some embodiments, the generation of the feedback signal can vary, in part, based on whether the detection and treatment system 201 is operating in an automatic mode or a guided mode. An example of operation in the automatic mode will be described relative to FIG. 4. An example of operation in the guided mode will be described relative to FIG. 5.

From block 305, the process 300 returns to the block 305, where the processor 203 causes the oxygenation sensor 216 to continue detecting an oxygenation status of ocular tissue as described previously. In various embodiments, the process 300 can continue until terminated by the operator or other suitable stop criteria is satisfied.

FIG. 4 illustrates an example of a process 400 for generating a feedback signal in an automatic mode, in accordance with certain embodiments of the present disclosure. In various embodiments, the process 400 can be performed, in whole or in part, as all or part of the block 305 of the process 300 of FIG. 3. For illustrative purposes, the process 400 will be described relative to the system 200 described relative to FIGS. 2A-B.

At block 401, the processor 203 receives a change in oxygenation status, such as the change in oxygenation status described relative to the decision block 304 of FIG. 3. At decision block 402, the processor 203 determines whether the change in oxygenation status indicates that ocular tissue corresponding to a current location of the probe tip 240, for example, is deoxygenated. If it is determined, at the decision block 402, that the ocular tissue is deoxygenated, the process 400 proceeds to block 403, where the processor 203 automatically causes (e.g., instructs) the treatment laser source 204A to transmit treatment laser light to the ocular tissue as described relative to FIGS. 2A-B. However, if it is determined, at the decision block 402, that the ocular tissue is not deoxygenated, the process 400 proceeds to block 404, where the processor 203 blocks utilization of the treatment laser source 204A to transmit treatment laser light. For example, the block 404 can include the processor 203 ignoring operator commands to transmit treatment laser light. After either block 403 or block 404, the process 400 ends.

It should be appreciated that the process 400 can be modified to suit a given implementation. In an example, blocking functionality can be performed under the conditions described relative to the block 404 without any automatic transmission occurring under the conditions described relative to the block 403. In another example, automatic transmission can be performed under the conditions described relative to the block 403 without any blocking being performed under the conditions described relative to the block 404. Other examples and variations will be apparent to one skilled in the art after a detailed review of the present disclosure.

FIG. 5 illustrates an example of a process 500 for generating a feedback signal in a guided mode, in accordance with certain embodiments of the present disclosure. In various embodiments, the process 500 can be performed, in whole or in part, as all or part of the block 305 of the process 300 of FIG. 3. For illustrative purposes, the process 500 will be described relative to the system 200 described relative to FIGS. 2A-B.

At block 501, the processor 203 receives a change in oxygenation status, such as the change in oxygenation status described relative to the decision block 304 of FIG. 3. At decision block 502, the processor 203 determines whether the change in oxygenation status indicates that ocular tissue corresponding to a current location of the probe tip 240, for example, is deoxygenated.

If it is determined, at the decision block 502, that the ocular tissue is deoxygenated, the process 500 proceeds to block 503, where the processor 203 signals the operator to manually transmit treatment laser light to the ocular tissue as described relative to FIGS. 2A-B. For example, the processor 203 can initiate visual, auditory, and/or haptic feedback to the operator as described relative to FIGS. 2A-B. In some embodiments, the processor 203 may thereafter receive an operator command to transmit the treatment laser light to the ocular tissue, in response to which the processor 203 may cause (e.g., instruct) the treatment laser source 204A to transmit the treatment laser light to the occur tissue. However, if it is determined, at the decision block 502, that the ocular tissue is not deoxygenated, the process 500 proceeds to block 504, where the processor 203 blocks utilization of the treatment laser source 204A to transmit treatment laser light. For example, the block 504 can include the processor 203 ignoring operator commands to transmit treatment laser light. After either block 503 or block 504, the process 500 ends.

It should be appreciated that the process 500 can be modified to suit a given implementation. In an example, blocking functionality can be performed under the conditions described relative to the block 504 without any signaling to the operator being performed under the conditions described relative to the block 503. In another example, operator signaling can be performed under the conditions described relative to the block 503 without any blocking being performed under the conditions described relative to the block 504. Other examples and variations will be apparent to one skilled in the art after a detailed review of the present disclosure.

FIG. 6 illustrates an example of an operating environment 600 for performance of a surgical procedure, such as treatment of ischemic tissue, according to embodiments of the present disclosure. As shown, the operating environment 600 further includes a surgeon 610, a patient 612, as well as a plurality of surgical systems and devices, such as a surgical console 620, a microscope system 624, and a display 626. The microscope system 624 can include binoculars 628. The surgical console 620 can operate as described, for example, relative to the surgical console 100 of FIG. 1. In certain embodiments, the surgical console 620 can include a detection and treatment system, such as the detection and treatment system 201 of FIGS. 2A-B.

The surgical console 620 includes a controller 604. In the example of FIG. 6, the controller 604 is integrated within the surgical console 620, where the controller 604 includes or refers to one or more processors and/or memory devices integrated within the surgical console 620. In certain other embodiments, the controller 604 is a stand-alone device or module that is in wireless or wired communication with, e.g., the surgical console 620, the microscope system 624, and other devices within the operating environment 600. In certain embodiments, the controller 604 refers to a set of software instructions that a processor associated with the surgical console 620 is configured to execute. In certain aspects, operations of the controller 604 may be executed partly by the processor associated with controller 604 and/or the surgical console 620 and partly in a public or private cloud.

In certain embodiments, the controller 604 can implement various aspects of a detection and treatment system such as the detection and treatment system 201 of FIGS. 2A-B. For example, the controller 604 can perform functionality of the processor 203 of FIG. 2B as described relative to FIGS. 2B and 3-4. In the context of the operating environment 600, the surgeon 610 can be presented real-time information via the binoculars 628 of the microscope system 624 and/or via the display 606 in any of the ways described previously. For example, the presented information can include a real-time retinal map of the type described relative to FIG. 2B. In addition, or alternatively, in some embodiments, the presented information can further include any collected hyperspectral and/or multispectral image data referenced relative to FIGS. 2A-B, such as multispectral images where deeper oxygen saturation of the ocular tissue is represented by generated mean scale values. In various embodiments, the surgeon 610 can thereby be provided actionable information, for example, for effective treatment of ischemic tissue.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

Within a claim, reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” The word “exemplary” is used herein to mean “serving as an example, instance, or illustration.” Any aspect described herein as “exemplary” is not necessarily to be construed as preferred or advantageous over other aspects.

What is claimed is:

1. A surgical system for treating ischemic tissue comprising:
an oxygenation sensor operable to detect an oxygenation status of a tissue based on illumination light reflected from the tissue;
a laser source operable to emit treatment laser light; and
a processor in communication with the oxygenation sensor and the laser source, wherein the processor is operable to generate a feedback signal related to utilization of the laser source in relation to the tissue based on the oxygenation status.

2. The surgical system of claim 1, wherein the generation of the feedback signal comprises:
determining, based on the oxygenation status, that the tissue is deoxygenated; and
responsive to the determining, automatically causing the laser source to transmit the treatment laser light to the tissue, the feedback signal comprising an instruction to the laser source.

3. The surgical system of claim 1, wherein the generation of the feedback signal comprises:
determining, based on the oxygenation status, that the tissue is deoxygenated; and
signaling an operator to manually transmit the treatment laser light to the tissue.

4. The surgical system of claim 3, wherein the signaling comprises initiating at least one of visual, auditory, or haptic feedback to the operator.

5. The surgical system of claim 3, wherein the processor is further operable to:
receive an operator command to transmit the treatment laser light responsive to the signaling; and
cause the laser source to transmit the treatment laser light to the tissue responsive to the operator command.

6. The surgical system of claim 1, wherein the generation of the feedback signal comprises:
determining, based on the oxygenation status, that the tissue is oxygenated; and
blocking utilization of the laser source responsive to the determining.

7. The surgical system of claim 1, further comprising:
an illumination source in communication with the processor and operable to emit the illumination light to the tissue; and
a surgical probe, the surgical probe including an optical fiber operatively coupled to the laser source and the illumination source, wherein the optical fiber is operable to transmit both the treatment laser light from the laser source and the illumination light from the illumination source.

8. The surgical system of claim 1, wherein the tissue comprises ocular tissue on a retinal surface of an eye.

9. A computer implemented method of treating ischemic tissue, the method comprising:
detecting, at an oxygenation sensor, an oxygenation status of a tissue based on illumination light reflected from the tissue; and
generating, at a processor, a feedback signal related to utilization of a laser source in relation to the tissue based on the oxygenation status.

10. The method of claim 9, wherein the generating the feedback signal comprises:
determining, at the processor, based on the oxygenation status, that the tissue is deoxygenated; and
responsive to the determining, at the processor, automatically causing the laser source to transmit treatment laser light to the tissue, the feedback signal comprising an instruction to the laser source.

11. The method of claim 9, wherein the generating the feedback signal comprises:
determining, at the processor, based on the oxygenation status, that the tissue is deoxygenated; and
signaling, by the processor, an operator to manually transmit treatment laser light to the tissue.

12. The method of claim 11, wherein the signaling comprises initiating at least one of visual, auditory, or haptic feedback to the operator.

13. The method of claim 11, further comprising:
receiving, by the processor, an operator command to transmit the treatment laser light responsive to the signaling; and
causing, by the processor, the laser source to transmit the treatment laser light to the tissue responsive to the operator command.

14. The method of claim 9, wherein the generating the feedback signal comprises:
determining, at the processor, based on the oxygenation status, that the tissue is oxygenated; and
blocking, by the processor, utilization of the laser source responsive to the determining.

15. The method of claim 9, wherein the tissue comprises ocular tissue on a retinal surface of an eye.
